(19) United States
(12) Patent Application Publication
(10) Pub. No.: US 2011/0060132 A1
(43) Pub. Date: Mar. 10, 2011

(54) SELF-CONTAINED, HIGH EFFICIENCY CELLULOSE BIOMASS PROCESSING PLANT

(76) Inventor: TED C. LEWIS, SIKESTON, MO (US)

(21) Appl. No.: 12/947,525

(22) Filed: Nov. 16, 2010

Related U.S. Application Data
(63) Continuation of application No. PCT/US2009/040365, filed on Apr. 13, 2009.
(60) Provisional application No. 61/055,222, filed on May 22, 2008.

(51) Int. Cl. C07K 1/14; B01J 19/10; B01J 19/12

(52) U.S. Cl. 530/412; 422/649; 422/128; 422/186.3; 422/186.01; 422/187

(57) ABSTRACT

A self-contained, high efficiency cellulose biomass processing plant includes sources of quantum-based wave energy to facilitate the dilute acid hydrolysis of hemi-cellulose and alpha-cellulose bond. The sources of quantum-based wave energy supply one or more of, and suitably a combination of ultrasonic waves, ultraviolet waves, magnetic waves and direct current to facilitate fracture of the intermolecular bonds. An integrated plant is also provided which combines the cellulose biomass processing plant with apparatus for converting high protein residue into a finished high grade protein product. Due to the high efficiency resulting from use of quantum-based wave energy, the plant uses less energy, and can be small and portable.

### CROSS-REFERENCE TO RELATED APPLICATIONS

This application is a Continuation Application of PCT/US2009/040365 filed Apr. 13, 2009, which claims priority to U.S. Ser. No. 61/055,222 filed May 22, 2008, herein incorporated by reference in their entirety.

### FIELD OF THE INVENTION

This invention is directed to a self-contained, high efficiency plant for processing cellulose biomass into fuel grade alcohol and a high-grade protein product. The processing plant can be portable and mobile, making it readily available at any location, to save the costs of transporting the cellulose biomass and/or the resulting products.

### BACKGROUND OF THE INVENTION

Volatile energy prices in recent years have caused increased interest in alternative sources of energy, including cellulose biomass. Primary sources of cellulose biomass in the United States include urban wood wastes, primary mill residues, forest residues, agricultural residues, and dedicated energy crops. Cellulose biomass plants, on the other hand, have been relatively large and incapable of being moved from their fixed locations. Cellulose biomass is often large and bulky, and can be expensive to transport to the processing plants. As a consequence, most available cellulose biomass is not processed into fuel grade alcohol or other products, but is instead laid to waste at or near its source. Conventional cellulose biomass processing plants also have drawbacks related to incomplete breakdown and conversion of the cellulose biomass into fuel grade alcohol and other by-products. One type of conventional plant relies on heat, pressure and residence time, in the presence of dilute acid, to perform the saccharification of the lingo-cellulosics in the biomass. Up to 50% by weight of the cellulose biomass is not saccharified the first time through, and needs to be separated and recycled. These factors contribute to the relatively large size and the fixed nature of conventional processing plants. One conventional biomass processing plant is described in U.S. Pat. No. 5,411,594, issued to Brelsford, the disclosure of which is incorporated by reference. Moreover, the by-product from conventional cellulose biomass processing plants is typically in the form of a heavy, wct, slurry that requires further conversion in order to provide useful end products. The by-product must typically be transported to another location for treatment, solids separation and further processing. The cost of transporting the by-product is high relative to the economics of the ultimate end products.

There is a need or desire for a cellulose biomass plant that operates with reduced cost and higher conversion of cellulose biomass to fuel grade alcohol and by-products. There is also a need or desire for a cellulose biomass processing plant that can be made small enough to be portable, and can be transported to the source of the biomass and/or the location where the end products are used.

### SUMMARY OF THE INVENTION

The present invention is directed to a self-contained, high efficiency cellulose biomass processing plant which a) achieves a much higher conversion percentage than conventional plants, b) requires less size and space than conventional plants, and c) can be made portable and transported to different locations. The plant includes a two-stage reactor for converting cellulose biomass into fuel grade alcohol and by-products. Each stage is equipped with one or more sources of quantum-based wave energy which aids in the chemical reaction and breakdown of cellulose biomass, and facilitates its conversion into fuel grade alcohol and by-products which are high in protein.

The sources of quantum-based wave energy include at least one, and suitably a combination of sources of ultrasonic waves, ultraviolet waves, magnetic waves (including electromagnetic and other magnetic waves), and direct current (nano-pulsed) waves. The sources of quantum-based wave energy facilitate the chemical reactions and conversion of cellulose biomass to such an extent that it becomes possible to substantially reduce the plant equipment size and residence time in each of the two stages while increasing the conversion percentage.

In the first stage, the fresh cellulose biomass feedstock is mixed with hot, pressurized dilute acid-water solution. The dilute acid-water solution, and some unhydrolyzed cellulose and lignin, may be supplied as a recycle stream from the second stage. The resulting aqueous cellulose biomass slurry is further heated to a reaction temperature of about 135-195° C., saturation pressure of about 45-200 psi, and a residence time of about 5-10 minutes. The one or more sources of quantum-based wave energy are applied to the slurry and help facilitate the hemicellulose hydrolysis reaction in the first stage.

The resulting hemicellulose hydrolysis reaction slurry is flashed to a reduced pressure and temperature, to minimize degradation of sugars. The slurry is then separated into a) a product solution, including hemi-cellulose hydrolysate pentose and hexose sugars and alpha-cellulose hydrolysate glucose sugar, and b) an unhydrolyzed cellulose solids residue, which is passed to the second stage as part of the alpha-cellulose hydrolysis feedstock for that stage.

In the second stage, the unhydrolyzed cellulosic solids residue from the first stage is combined with a recycled fraction of unhydrolyzed cellulosic solids residue from the second stage to provide an alpha-cellulose hydrolysis feedstock. The alpha-cellulose hydrolysis feedstock is mixed with fresh dilute acid-water solution to form an alpha-cellulose hydrolysis reaction slurry. The slurry is heated to a temperature of about 165-260° C. at a saturated pressure of about 100-200 psi, for a time of about 8-17 minutes. The one or more sources of wave energy are applied to help facilitate conversion of the alpha-cellulose hydrolysis reaction slurry into alpha-cellulose hydrolysate glucose sugar, dissolved in the dilute acid-water solution.

The resulting product slurry is flashed to a reduced pressure and temperature, to minimize glucose sugar degradation. The product slurry is then separated to recover a) surplus process heat as flashed steam, b) a hot alpha-cellulose hydrolysate sugar and dilute acid solution, and c) solids including high protein product residue with minimal or no unhydrolyzed alpha-cellulose. The solution can then be separated from the high protein product residue and recycled into the first stage reaction mixture. The present invention also includes an integrated plant which combines the cellulose biomass processing plant with apparatus for converting the high protein product residue into a finished high grade protein product. The integrated plant can be made portable, such as by designing the integrated plant on a platform with wheels or in modules transportable by a carrier. The portable integrated plant can be made small enough to transport it between different sources of cellulose biomass feedstock and/or different locations where the food grade alcohol product and/or the high grade protein product are used.

With the foregoing in mind, it is a feature and advantage of the invention to provide a self-contained, high efficiency cellulose biomass processing plant which, due to its higher efficiency, can be made smaller and easier to transport than conventional plants. It is also a feature and advantage of the invention to provide a self-contained, high efficiency cellulose biomass processing plant which is portable.

It is also a feature and advantage of the invention to provide an integrated plant which combines the cellulose biomass processing plant with apparatus for converting the high protein product residue into a useful high grade protein product, and which can be portable.

It is also a feature and advantage of the invention to provide improvements to the efficiency and operation of the cellulose biomass processing plant described in the above-identified Brelsford patent including reduced need for catalyst and process heat and reduced reactor size due to the added sources of quantum-based wave energy. The same volume of biomass can now be processed using a reactor that is not more than half of the previous size.

### BRIEF DESCRIPTION OF THE DRAWINGS

FIG. 1 schematically illustrates a self-contained, high efficiency cellulose biomass processing plant of the invention.

FIG. 2 schematically illustrates an integrated plant which combines the cellulose biomass processing plant of the invention with apparatus for converting the high protein product residue into a finished high grade protein product.

### DETAILED DESCRIPTION OF THE INVENTION

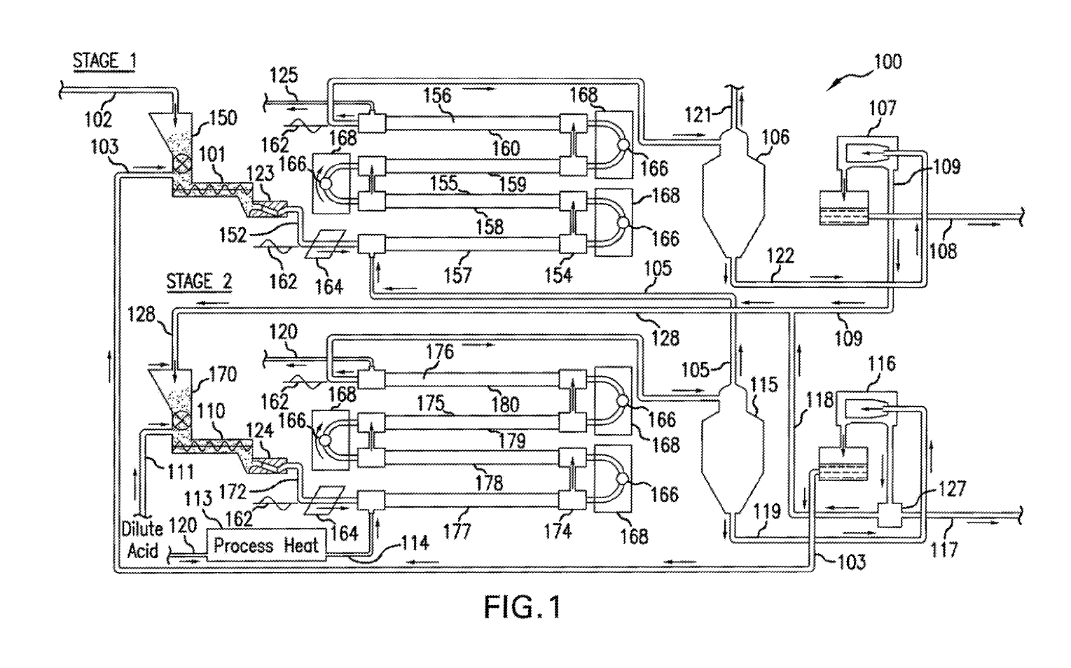

FIG. 1 schematically illustrates a self-contained, high efficiency cellulose biomass processing plant 100 of the invention. The schematic diagram of FIG. 1 combines improvements of the invention with a two-stage system including two double-tube heat exchanger plug flow reactors and flash tank subsystems in series, as described in U.S. Pat. No. 5,411,594 to Brelsford, the disclosure of which is incorporated by reference. The improvements described below enable a higher efficiency, smaller size and portability of the plant 100.

The plant 100 includes a first stage 1 and a second stage 2. In the first stage 1, cellulose biomass feedstock, typically in the form of distillers wet grains (DDG's), is conveyed through an inlet 102 to a rotary feeder 150. A solution of dilute acid enters the plant 100 through inlet 103, and is mixed with the cellulose biomass feed in the slurry mixer 101 below rotary feeder 150.

The cellulose biomass feed may be derived from various sources of plant-based cellulose, including without limitation wood from trees, such as pine soft wood and other kinds of soft wood and hard wood; food plants, such as corn, wheat, barley and soybeans; grasses; recycled cellulose products such as paper or cardboard; and other sources of cellulose. The dilute acid solution may be an aqueous solution including about 0.75-2.5% by weight, suitably about 1.5-2.2% by weight acid. Suitable acids include without limitation sulfuric acid, nitric acid, hydronium ionic acid, and the like. The slurry mixture of cellulose biomass feed and dilute acid solution may include about 5-75% by weight cellulose biomass solids, suitably about 8-55% by weight cellulose biomass solids. The dilute acid solution, when fed via inlet 103, may also include some recycled cellulose biomass solids which are included in the foregoing weight percentages of cellulose biomass solids.

The dilute acid solution may be preheated, and/or the slurry mixer 101 may be heated, in order to partially reach the desired stage 1 reaction temperature of about 135-195° C., suitably about 150-175° C., and the desired saturation pressure of about 45-200 psi, suitably about 120-190 psi. The slurry mixture is conveyed from mixer 101 via slurry pump 123 into the inner tube 152 of the stage 1 plug flow reactor 154. The plug flow reactor 154 includes the inner tube 152, where the stage 1 reaction takes place, and interconnected outer tubes 156, which define heat exchanger 155 and supply the necessary heating fluid to maintain the desired stage 1 reaction temperatures and pressures indicated above.

As shown in FIG. 1, the heat exchanger 155 includes four interconnected outer tubes 156 for supplying heat exchanger fluid. The inner reactor tube 152 passes lengthwise through each of the outer tubes 156, and thus makes four passes 157, 158, 159 and 160 through the heat exchanger 155. The heat exchanger 155 can be supplied with a suitable heating fluid, for example superheated steam, which is fed through the outer tubes 156 to heat the inner reactor tube 152. In accordance with the invention, the inner reactor tube 152 is also provided with one or more sources of quantum-based wave energy. The sources of quantum-based wave energy illustrated in FIG. 1 include direct current sources 162, photonic energy (ultraviolet wave) sources 164, ultrasonic wave sources 166 and electromagnetic wave sources 168.

A direct current source 162 and a photonic energy source 164 are each supplied to the inner reactor tube 152 before it enters the first pass 157 through heat exchanger 156. The direct current from source 162 is supplied at about 0.2 to about 3.0 amperes. The direct current energizes the polar moieties of the carboxyl groups in the cellulose, and facilitates fracturing of the molecular bonds and breakdown of the cellulose. The direct current can be provided from any suitable source, including without limitation an AC-DC transformer or battery, available from Hoefer, Inc. The photonic (ultraviolet) wave energy from source 164 is supplied in the ultraviolet range at about 10¹⁵ to about 10¹⁶ hertz. The photonic wave energy assists in energizing and breaking the alpha-cellulose bonds, which are otherwise more difficult to break. The photonic energy can be provided from any suitable source, including without limitation a halogen bulb ultraviolet source of Nd-Yag laser, available from Photochemical Reactors, Ltd.

An ultrasonic wave source 166 and an electromagnetic wave source 168 are each supplied to the inner reactor tube 152 between the first pass 157 and the second pass 158, between the second pass 158 and the third pass 159, and between the third pass 159 and the fourth pass 160 through heat exchanger 156. The ultrasonic wave energy helps prevent and/or reverse any physical agglomeration of the cellulose biomass and its sub-components and reaction product components. Specifically, ultrasonic waves induce micro-cavitations in the reactor liquid, which in turn generate micro-pressure waves which cause constantly changing orthothrombic distortions in the cellulose crystal structure. The micro-pressure waves generate wave energy that is absorbed by chemical bonds through wave-form resonance, resulting in increased bond length, instability and breakage of the cellulose bonds. The ultrasonic waves are supplied at an acoustic frequency of about 20 KHz to about 60 KHz using any suitable ultrasonic transducer, for example a tube resonator transducer, available from Telsonic.

The electromagnetic energy bends bond angles in the polar moieties of the carboxyl groups in the cellulose. The bending of the bond angles increases the effectiveness of ionopheresis, and facilitates fracturing of the molecular bonds. Specifically, the carboxyl bond-angle distortion produced in a single molecule by an electromagnetic field can exceed 0.5%, significantly increasing its vulnerability to dilute-acid cellulose hydrolysis. The electromagnetic energy is supplied at low frequency at a field strength of about 20-1000 gauss. Any suitable source of electromagnetic energy can be employed, for example a permanent magnet or electromagnet, available from Eriez.

A second direct current source 162 is supplied to the inner reactor tube 152 following the fourth pass 160, at about 0.2 to about 3.0 amperes. This causes further bond energizing and destabilizing of the carboxyl groups in the cellulose. While in the reactor tube 152, the cellulose biomass is converted to a slurry of hydrolyzed hemicellulose and a product solution. The sources of wave energy, combined with the reactor heat and pressure, allow the reaction to proceed through the first stage 1 at a reactor residence time of about 1.5-4.5 minutes, while achieving a conversion percentage (hydrolysis of cellulose bonds) of about 85-100% based on the dry weight of the cellulose biomass feed.

The resulting hemicellulose hydrolysis reaction slurry is passed into a flash tank 106 where it is flashed to a reduced temperature of about 35-65° C. and a reduced pressure of about ambient atmospheric pressure. Flashed steam exits flash tank 106 via outlet 121 and can be used in the second stage 2 to preheat the dilute acid solution. The reaction slurry passes via conduit 122 into a separator 107, which separates the slurry into a product solution, including hemi-cellulose hydrolysate pentose and hexose sugars and alpha-cellulose hydrolysate glucose sugar, and an unhydrolyzed cellulose solids residue. The product solution exits the separator 107 and the plant 100 through outlet 108. The unhydrolyzed cellulose solids residue is conveyed by conduit 109 into the second stage 2 of the plant 100.

Referring to FIG. 1, conduit 109, carrying the unhydrolyzed cellulose solids residue from stage 1, converges with conduit 118, carrying a recycled fraction of unhydrolyzed cellulose solids residue from stage 2. At this stage, the unhydrolyzed cellulose bonds are mostly alpha-cellulose, with most of the hemi-cellulose bonds having been hydrolyzed in stage 1. The combined conduit 128 feeds the unhydrolyzed cellulose solids residue into the stage 2 rotary feeder 170 followed by slurry mixer 110, which combines the unhydrolyzed cellulose solids residue with dilute acid solution from inlet 111. The dilute acid solution may be an aqueous solution including about 1.75-2.5% by weight, suitably about 2.0-2.25% by weight acid. Suitable acids include without limitation sulfuric acid, nitric acid, hydronium ionic acid and the like. The dilute acid entering stage 2 inlet 111 may be of the same type and concentration as the dilute acid entering stage 1 inlet 103. The slurry mixture in mixer 110 may include about 5-75% by weight unhydrolyzed cellulose solids, suitably about 8-55% by weight.

The dilute acid solution may be preheated, and/or the slurry mixer 110 may be heated, in order to help achieve the desired stage 2 reaction temperature of about 165-260° C., suitably about 180-225° C., and the desired saturation pressure of about 100-250 psi, suitably about 180-220 psi. As with stage 1, much of the desired heat and pressure will be accomplished in the plug flow reactor. Any heating prior to the plug flow reactor may only bring the temperature and pressure of the slurry part of the way to the desired levels.

The slurry mixture is conveyed from mixer 110 via slurry pump 124 into the inner tube 172 of the stage 2 plug flow reactor 174. The plug flow reactor 174 includes the inner tube 172, where the stage 2 reaction takes place, and interconnected outer tubes 176, which define heat exchanger 175 and supply the necessary heating fluid to maintain the desired stage 2 reaction temperatures and pressures indicated above. As shown in FIG. 1, the stage 2 heat exchanger 175 includes four interconnected outer tubes 176 for supplying the heat exchanger fluid. The inner reactor tube 172 passes lengthwise through each of the outer tubes 176, and thus makes four passes 177, 178, 179 and 180 through the heat exchanger 175. The heat exchanger 175 can be supplied with a suitable heating fluid, for example superheated steam, which is fed through the outer tubes 176 to heat the inner reactor tube 172. In accordance with the invention, the inner reactor tube 172 is also provided with one or more sources of quantum-based wave energy. The sources of wave energy indicated in FIG. 1 include direct current sources 162, photonic energy (ultraviolet wave) sources 164, ultrasonic wave sources 166 and electromagnetic wave sources 168.

A direct current source 162 and a photonic energy source 164 are each supplied to the inner reactor tube 172 before it enters the first pass 177 through the heat exchanger 176. The direct current from source 162 is supplied at about 0.2 to about 3.0 amperes. The photonic (ultraviolet) wave energy from source 164 is supplied at about 10¹⁵ to about 10¹⁶ hertz. The apparatus for supplying these energy sources to the stage 2 reactor 174 may be the same as indicated for the stage 1 reactor 154. An ultrasonic wave source 166 and an electromagnetic wave source 168 are each supplied to the inner reactor tube 172 between the first pass 177 and the second pass 178, between the second pass 178 and the third pass 179, and between the third pass 179 and the fourth pass 180 through heat exchanger 176. The ultrasonic waves are supplied at an acoustic frequency of about 20 KHz to about 160 KHz. The electromagnetic energy is supplied at a low frequency at a field strength of about 20 to about 1000 gauss. The apparatus for supplying these energy sources to the stage 2 reactor 174 may be the same as indicated for the stage 1 reactor 154.

A second direct current source 162 is supplied to the inner reactor tube 172 following the fourth pass 180, at about 0.2 to about 3.0 amperes. The sources of wave energy, combined with the reactor heat and pressure, allow the reaction to proceed through the second stage 2 at a reactor residence time of about 7.0 to about 14 minutes, while achieving an overall conversion percentage of about 85-100% based on the dry weight of cellulose biomass feed entering the first stage 1. The stage 2 reactor 174 converts the substantially alpha-cellulose hydrolysis feedstock entering the second stage 2 into a product slurry that includes alpha cellulose hydrolysate glucose sugar, dissolved in the dilute acid-water solution, and a solid product fraction.

The product slurry is passed into a flash tank 115 where it is flashed to a reduced temperature of about 55-85° C. and a reduced pressure of about ambient atmospheric pressure. Flashed steam exits flash tank 116 via outlet 105 and can be recycled for use as heating fluid for stage 1 heat exchanger 156, as shown in FIG. 1. The remainder of the product slurry passes via conduit 119 into separator 116, which separates the remaining slurry into a solution, including alpha-cellulose hydrolysate glucose sugar in dilute acid, and the solid product fraction. The solution exits separator 116 via line 103 and is recycled back into the stage 1 slurry mixer 101 as shown. Due to the conversion efficiency of the plant 100, the solid product fraction is composed entirely or substantially of a high protein product residue that includes minimal or no unhydrolyzed alpha-cellulose. The high protein product residue exits the separator 116 through outlet 117, for further processing into a high grade, high protein product.

The plant 100 includes a process heat supply 113 that initially transfers heat to a heating fluid, such as a high pressure steam, that is input via line 120 and conveyed via line 114 into the heat exchanger 175 of stage 2 reactor 174. After passing through heat exchanger 175, the heating fluid is recycled via line 120 and is again heated using heat supply 113. The heating fluid for the stage 1 reactor 154 is supplied from steam which is flashed from stage 2 flash tank 115 via line 105, which leads to heat exchanger 155 of the stage 1 reactor. Additional heat from a heating source (not shown) may be required to raise that steam to the desired temperature and pressure. Other heating apparatus and techniques can also be employed without departing from the invention.

Because of the improvements including the sources of wave energy, the cellulose biomass processing may achieve more than about 80%, or more than about 85%, or more than about 90%, or more than about 95%, or about 100% hydrolysis of the hemi-cellulose and alpha-cellulose bonds, thus minimizing the amount of unhydrolyzed cellulose in the residue exit stream 117. This facilitates better conversion of the high protein residue into useful high protein products.

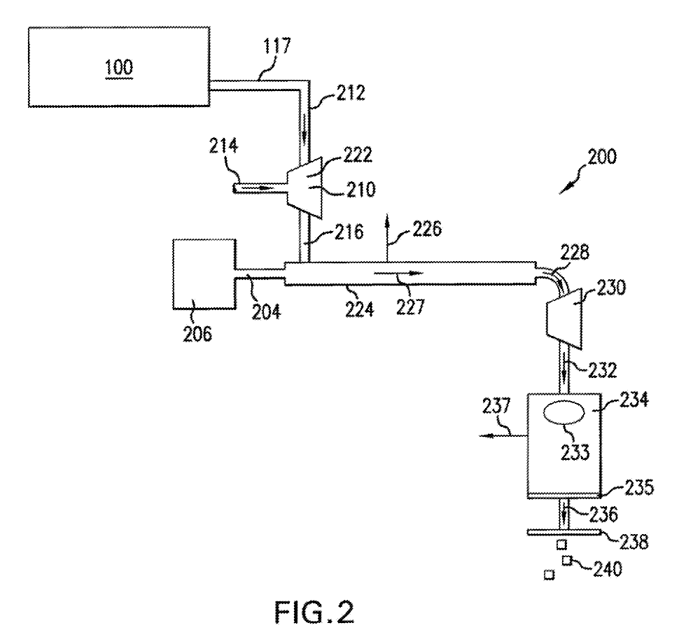

FIG. 2 schematically illustrates an integrated plant 200 that combines the cellulose biomass processing plant of FIG. 1 with apparatus for converting the high protein product residue into a finished high grade high protein product. In FIG. 2, the entire cellulose biomass processing plant 100 of FIG. 1 is represented as a single block 100, for purposes of simplicity. The high protein residue that exits the plant 100 via outlet 117 is fed via inlet 212 to a mixer hopper 210. This residue may include a plurality of ingredients selected from grain residue, dissolved sugars and proteins, residual alcohol, water, carbon dioxide, yeast, enzymes, and other by-products and ingredients. The residue may be combined in the mixer hopper 210 with flavor enhancers, nutrients, texture modifiers, precipitating agents and other additives fed to the mixer hopper via inlet 214, to provide a combined residue mixture 222. Exemplary flavor enhancing ingredients include without limitation grain extracts, synthetic flavors, mineral salts, and combinations thereof. Exemplary nutrients include without limitation vitamins, minerals, proteins, and combinations thereof. Exemplary texture modifiers include without limitation grain extracts, proteins, synthetic additives, and combinations thereof. Exemplary precipitating agents include organic and inorganic precipitants and combinations thereof.

The residue mixture is fed from the mixer hopper via outlet 216 into a compression apparatus which, in this embodiment, can be a twin screw extruder 224 driven by a drive motor 206 and drive shaft 204. Twin screw extruders are designed to accommodate a variety of screw configurations tailored for specific purposes. Twin screw extruder 224 is designed for mixing and compression of the ingredients, and includes a vacuum port at the location of arrow 226. The vacuum port communicates with one or more vacuum pumps (not shown) to devolatilize and remove large amounts of water, in the form of water vapor, from the residue slurry. As the water vapor is removed, the residue mixture experiences decreasing viscosity and higher pressure as it is conveyed forward in the direction of arrow 227. The compression apparatus may reduce the liquid content in the residue mixture from a starting content which can be greater than 80% by weight, to a much reduced liquid content of about 3-8% by weight.

The compressed residue mixture passes from twin screw extruder 224 through channel 228 and pressure pump 230, which raises the pressure mixture to between about 250 and about 3000 psi. The residue mixture then passes through channel 232 into a drying apparatus, illustrated as a flash drying chamber 234 and enters a compression zone 233 in the chamber 234. While in the compression zone 233, or immediately prior thereto, the residue mixture is combined with compressed, heated gas such as air or carbon dioxide, at a pressure of about 250 to about 3000 psi and a temperature of about 65° C. to about 180° C. The subsequent release of pressure as the residue mixture leaves compression zone 233 results in rapid decompression and explosive “flash” drying in the chamber 234. The residual water vapor leaves chamber 234 through outlet 237. The resulting dried high grade protein product, which has a moisture content of less than about 3% by weight, passes through a shaping apparatus, illustrated as extrusion plate 235 in the form of one or more strands 236.

The strands 236 can be granulated using any suitable blade assembly, pelletizer or other granulation apparatus 238, to form pellets 240 of high grade protein product. The high grade protein product can be packaged and stored, or conveyed for immediate use. Other suitable apparatus for converting the high protein product residue into a high grade protein product may also be employed, and may be combined with cellulose biomass processing plant 100 to form an integrated plant. Such other apparatus will generally include a compression apparatus, a drying apparatus, a shaping apparatus and a granulation apparatus, but may perform some of the corresponding steps in a different order than illustrated in FIG. 2.

The integrated plant 200 of FIG. 2 performs the drying step in flash drying chamber 234 before the shaping step, which is largely performed as the dried compressed residue passes through extrusion plate 235. The explosive flash drying results in a high grade protein product having a low moisture content which can be less than about 2% by weight, suitably less than about 1% by weight, or less than about 0.5% by weight, or less than about 0.2% by weight, or less than about 0.1% by weight, or less than about 0.05% by weight, or less than about 0.02% by weight. The low moisture content enhances the storage stability of the high grade protein product by alleviating moisture-induced spoilage.

The cellulose biomass processing plant 100, and/or the integrated plant 200, can be made portable by affixing the plant to a steel platform or other suitable support device. The platform can be provided with wheels, and can be provided with a hitch for pulling with a vehicle. The portability of the cellulose biomass processing plant 100 and/or the integrated plant 200 is made possible by the higher plant efficiency and smaller size requirements that result from the use of wave energy to facilitate hydrolysis of the hemi-cellulose and alpha-cellulose bonds.

In addition to increasing plant efficiency and reducing plant size, the use of quantum-based wave energy dramatically reduces the energy consumption required to operate the plant. The portable nature of the plant allows it to be transported to various sources of cellulose biomass, thereby eliminating the cost of transporting cellulose biomass. The plant can be made large or small, depending on the nature of the operation. The plant can be made large for high volume commercial use, or can be made small for lower volume use by private individuals.

The embodiments of the invention described herein are exemplary. Various modifications and improvements can be made without departing from the spirit and scope of the invention. The scope of the invention is indicated in the appended claims, and all changes that fall within the meaning and range of equivalents are intended to be embraced therein.

What is claimed is:

1. A cellulose biomass processing plant, comprising:
a first stage including a first reactor, a source of heat for the first reactor, and one or more first stage sources of energy in communication with the first reactor;
a second stage including a second reactor, a source of heat for the second reactor, and one or more second stage sources of energy in communication with the second reactor;
wherein the first stage receives cellulose biomass feed and an acid solution which causes hydrolysis of cellulose of the cellulosic biomass feed, and the second stage receives unhydrolized cellulose from the first stage and an acid solution and causes further hydrolysis of the unhydrolized cellulose from the first stage.

2. The cellulose biomass processing plant of claim 1 wherein the one or more first stage sources of energy being configured so as to enhance yield for the hydrolysis of cellulose of the cellulosic biomass feed and wherein the one or more second stage sources of energy being configured so as to enhance yield for the hydrolysis of the unhydrolized cellulose from the first stage such that the cellulose biomass processing plant achieves more than about 80 percent hydrolysis of the cellulose.

3. The cellulose biomass processing plant of claim 1 wherein the first reactor is a continuous flowing reactor and the second reactor is a continuous flowing reactor.

4. The cellulose biomass processing plant of claim 1, wherein the one or more first stage sources of energy is selected from the group consisting of sources of photonic energy, ultrasonic energy, ultraviolet energy, magnetic energy, direct current, and combinations thereof.

5. The cellulose biomass processing plant of claim 1, wherein the one or more second stage sources of energy is selected from the group consisting of sources of photonic energy, ultrasonic energy, ultraviolet energy, magnetic energy, direct current, and combinations thereof.

6. The cellulose biomass processing plant of claim 1, further comprising apparatus for receiving high protein residue from the second stage and converting it into a high grade protein product.

7. The cellulose biomass processing plant of claim 6, wherein the apparatus for receiving and converting the high protein residue comprises a compression apparatus, a shaping apparatus, a drying apparatus and a granulation apparatus.

8. The cellulose biomass processing plant of claim 1, further comprising a platform with wheels for transporting the plant.

9. The cellulose biomass processing plant of claim 1 wherein the cellulose is selected from the set consisting of wood, corn, wheat, barley, soybeans, grasses, and recycled cellulose products.

10. A process for treating cellulose biomass feedstock comprising the steps of:
combining the cellulose biomass feedstock with an acid solution to form a slurry;
heating the slurry;
passing the slurry through a reactor;
supplying the reactor with one or more sources of energy to facilitate hydrolysis of hemi-cellulose and alpha-cellulose and form a product slurry; and
separating solid and liquid components of the product slurry.

11. The process of claim 10, wherein the reactor comprises a first stage and a second stage, and one or more sources of energy are supplied to each of the stages of the reactor.

12. The process of claim 11 wherein each of the one or more sources of energy are selected from a set consisting of sources of photonic energy, ultrasonic energy, ultraviolet energy, magnetic energy, direct current, and combinations thereof.

13. The process of claim 12 wherein the one or more sources of energy are supplied at a plurality of locations in the first stage of the reactor and a plurality of locations in the second stage of the reactor.

14. The process of claim 10, wherein the product slurry is separated into a liquid stream and a high protein residue, further comprising the steps of compressing, shaping, drying and granulating the high protein residue to form a high grade protein product.

15. The process of claim 10 wherein the cellulose is selected from the set consisting of wood, corn, wheat, barley, soybeans, grasses, and recycled cellulose products.

16. A cellulose biomass processing plant, comprising:
a first stage including a first reactor, a source of heat for the first reactor, one or more first stage sources of energy in communication with the first stage reactor, a flash tank in communication with the first stage reactor, and a separator in communication with the flash tank;
wherein the one or more first stage sources of energy are provided in communication with a plurality of locations in the first stage reactor;
a second stage including a second reactor, a source of heat for the second reactor, one or more second stage sources of energy in communication with the second stage reactor, a flash tank in communication with the second stage reactor and a separator in communication with the flash tank;
wherein the one or more second stage sources of energy are provided in communication with a plurality of locations in the second stage reactor.

17. The cellulose biomass processing plant of claim 16, wherein the one or more sources of energy are supplied at a plurality of locations in the first stage of the reactor and a plurality of locations in the second stage of the reactor consisting of sources of photonic energy, ultrasonic energy, ultraviolet energy, magnetic energy, direct current, and combinations thereof.

18. The cellulose biomass processing plant of claim 16 wherein the cellulose biomass processing plant is configured to achieve more than about 80 percent hydrolysis of cellulose.
